(19) United States
(12) Patent Application Publication
(10) Pub. No.: US 2016/0302667 A1
(43) Pub. Date: Oct. 20, 2016

(54) COHERENT ELECTROMAGNETIC WAVES AID RECONCILIATION

(71) Applicant: Joel Steven Goldberg, Hillsborough, NC (US)

(72) Inventor: Joel Steven Goldberg, Hillsborough, NC (US)

(21) Appl. No.: 15/191,673

(22) Filed: Jun. 24, 2016

(51) Int. Cl. A61B 5/00; A61B 5/04; A61B 5/0476

(52) U.S. Cl. CPC A61B 5/0006; A61B 5/0476; A61B 5/04012; A61B 5/7257

(57) ABSTRACT

Reconciliation of past events is beneficial for resolution of conflict but not essential to resolve geographic, political and economic conflicts that can cease violence. Past events are immutable in spacetime and interpretation of past events may be different for different observers. These events that differ among observers may lead to very different outcomes associated with the event because the activity of the brain is chaotic. Although humans have an innate desire to reconcile past events, resolution of conflicts should be initially concerned with the present and future. Reconciliation is a long and arduous process. In this invention electromagnetic waves that are coherent with natural waves generated by the brain of those in conflict are broadcast through air or wire into areas of conflict to aid reconciliation. Peace may be more easily obtainable with this invention, but this invention will not in itself solve the problem of reconciliation.

### CROSS-REFERENCES TO RELATED APPLICATIONS

None

### FEDERALLY FUNDED RESEARCH

Not applicable

### BACKGROUND OF THE INVENTION

Conflicts among individuals, groups and countries are commonly resolved through negotiations. Conflicts that escalate among countries can lead to war.

The two greatest preventable threats to humanity from conflict are: 1) weapons of mass destruction launched by leaders of a rogue nation and 2) weapons of mass destruction launched by a nation in conflict because mutually assured destruction is not likely. (Goldberg, 2016b) The possibility of weapons of mass destruction launched by accident will exist as long as the weapons are present in the arsenals of countries. Those who are not physically impaired from use of weapons of mass destruction will suffer life-long psychological trauma. (Goldberg, 2008) In contrast to chemical and biological weapons, detonation of nuclear weapons will destroy infrastructure in addition to killing and crippling life on earth.

During conflict, skilled professionals, either appointed or elected, negotiate for their country to settle disputes. Settlement of conflicts between two parties can be resolved as lose-lose, win-lose or win-win outcomes. (Colemen, 2015) Lose-lose resolutions are the result of mutual compromise where both parties lose something. Win-lose resolutions occur when the conflict resolution is competitive and one party wins with and the other party loses. Win-win resolutions are the result of cooperative rather than competitive negotiations when both parties receive more than they expect from the negotiation process and further negotiations are unlikely to improve outcome. Win-win resolutions are more likely to be sustainable and are more easily accepted by constituents.

Impediments to negotiations between two parties are numerous and include:
1. Significant power differences between the two parties
2. Unilateral or mutual distrust
3. Unilateral or mutual disrespect
4. Unilateral or mutual humiliation
5. Unresolvable fear, anger and hatred
6. Difficult communication
7. Lack of creativity
8. Biases that cannot be overcome

Win-win resolutions of conflict do not necessarily lead to peace. Win-win resolutions define the economic, geographic and political terms of settlement, but reconciliation between the parties may be required for lasting peace. Unlike economic, geographic and political agreements, reconciliation takes place in the minds of both the negotiators and their constituents.

In conflicts, reconciliation of past events of wrong doing are encoded in the minds of those responsible for such actions and in the minds of those that have been hurt. These past events cause pain that may be difficult to alleviate. Reconciliation requires that the pain from the past events be ameliorated by forgiveness or justification. In contrast to negotiated conflict resolution, reconciliation is a slow mental process that may take generations to resolve.

It is well known that perspective, or frame of reference, determines the perception of events. Reconciliation is a much slower and a more difficult process compared to negotiations that resolve economic, geographic and political disputes for the following reasons:
1. The past are events in spacetime and the information from the events is transmitted to the brain, encoded in spacetime and formulated in the mind. The information of events is transmitted through the visual system via electromagnetic waves, through the auditory system by compression waves and through the sensory system including taste, smell and touch by activation of receptors. All information is encoded to the brain through action potentials. The spacetime coordinates of events in the brain are different for each observer and can change the trajectory of chaotic brain activity. (FIGS. 1&2) Therefore, reconstructing of past events is different for each observer and this makes reconciliation more difficult.
2. The amount of pain that each person experiences from events is variable. Some experience less pain and presumably can more easily attain reconciliation vs. those who experience intense pain that may be more difficult to reconcile.
3. Moral justification of events may be different for each observer. World religions have very differing views on how reconciliation is achieved.
4. Enthusiasm or desire to reconcile may be different between observers. Reconciliation may not be an essential part of moral, religious or philosophical thought for some observers.
5. Thoughts and feelings may interfere with reconciliation.

Many ritual-like behaviors have been shown to improve conflicted relationships and aid in reconciliation. These include handshaking, sharing a meal, sharing a walk, sharing in prayer and sharing a peace pipe. In history these ritual-like behaviors have led to important breakthroughs and reconciliation. For example: President Jimmy Carter brought Anwar Sadat and Menachem Begin to the cemetery at Gettysburg, Pa. to deliberate when there was an impasse in peace talks between these leaders of Egypt and Israel. Using the power of shared experiences, President Ronald Reagan invited Mikhail Gorbachev to the Reagan's California ranch where both leaders rode horses. These ritual-like behaviors aided negotiations and reconciliation.

Practiced throughout the world in different cultures, meditation is associated with improvement in health and well-being. Some forms of meditation include mindfulness, transcendental, relaxation response and prayer. A common electrophysiologic response to some forms of meditation is EEG coherence which is associated with a peaceful state of mind. (FIG. 3)

Previously it was shown that work from external electromagnetic waves can change the electrical activity of a thermodynamic model of a nervous system. In the same model, interference patterns were observed when external electromagnetic waves superposition with waves generated in this electrochemical system. (Goldberg, 2015, 2016a) Also previous work has shown that the area of least impedance of the skull lies over the pterion-squamous temporal bone junction where stimulation by extremely low frequency electromagnetic waves produces phosphenes as evidence of cortical stimulation. (Goldberg, 2016b). External electromagnetic waves are predicted to preferentially affect the temporal lobe within which lies the amygdala and hippocampus. The most likely conclusion from these observations is that there are electromagnetic interferences in the form of waves between the environment and the brain. Therefore, it is proposed that broadcasting coherent electromagnetic waves will aid reconciliation by producing a more peaceful state of mind.

### DESCRIPTION OF THE DRAWING

FIG. 1 is a light cone that describes past, present and future events in two dimensional spacetime. Label 1 is an observer. Label 2 is an observer. Label 3 is a past event that is encoded in the brain of each observer with different spacetime coordinates.

FIG. 2 is a Minkowski diagram in two dimensional spacetime showing simultaneous events with different spacetime coordinates. Label 1 is the location of time for both events. Simultaneity of events is relative to the reference frame.

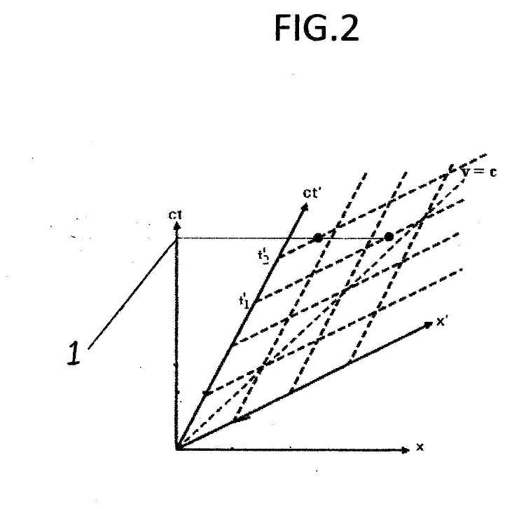

FIG. 3 shows coherent electrical activity associated with peaceful states during meditation. Label 1 is normal EEG activity. Label 2 is coherent EEG activity during meditation. (Ferguson, 1975)

FIG. 4 is a flow diagram of the invention.

### DETAILED DESCRIPTION OF THE INVENTION

It is proposed during conflict negotiations, that reconciliation of past events can be separated from conflicts of present and future economic, geographic, and political disputes and cessation of violence can be achieved without full reconciliation.

Present theories of conflict resolution propose reconciliation of past events to include admission of wrongdoing and forgiving as part of the resolution of a conflict. In order for the past to be reconciled, both parties must reconstruct the events in their minds in spacetime. An event in the mind may be defined as a manifestation of the elements of a configuration space consisting of synaptic and neural activity and time. A configuration space is the vector representation of the coordinates of these elements.

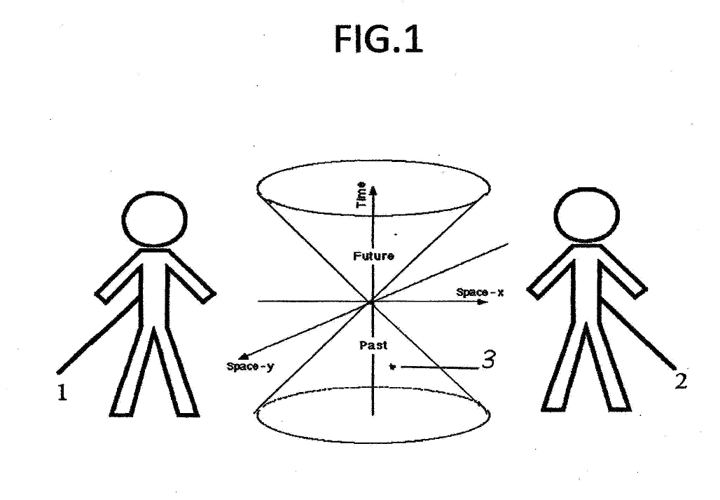

A past event is a point in spacetime that cannot be changed by present and future actions. Humans have an innate desire to reconcile the past, but cessation of violence during conflicts can be achieved without full reconciliation. Complete reconciliation can only be achieved in the minds of those in conflict. Observers in different reference frames reconstruct simultaneous past events in the mind with different spacetime coordinates in the brain. The events are perceived differently because the spacetime coordinates that describe the past events are not the same. The past events are different for each observer as depicted by a light cone with two observers and the Minkowski spacetime diagram of simultaneous events relative to the observer. (FIGS. 1&2) These differences in observation of an event may become magnified, changing the trajectory of the chaotic activity of the brain.

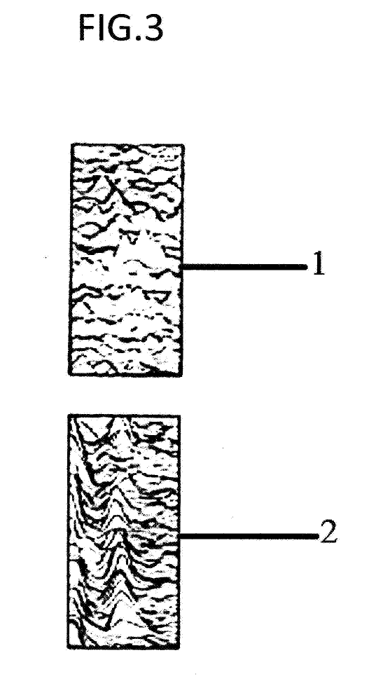

It has been known for some time that thoughts can change the electrical activity of the brain as measured by EEG or equivalents. Some examples include the changes in EEG coherence during meditation states. (FIG. 3) In thermodynamic terms, thoughts of meditation do work on a system comprising neuronal activity and synapses that is separate from the system that generates thoughts. The transfer of internal energy to the system is reflected in changes in electrical activity of the brain. Since thoughts can produce EEG coherence, external electromagnetic waves at the proper frequency, wavelength, conformation and time can produce coherence that can be measured by EEG or its equivalent. Coherence is associated with a more peaceful state of mind.

The frequencies of these coherent waves are likely to be in the range of 5-40 Hz. The conformation of these waves is likely to be unipolar and similar to the natural conformation of waves of humans.

Unlike the physical universe, activities in the mind do not always follow the laws of physics. In the mind the past can be mutable and time can travel in either direction. The future is limitless constrained by the imagination unlike the spacetime constraints of a light cone. If information is stored in waves in the mind then multiple waves can exist in the same space in the brain. These properties of mind may make reconciliation more difficult in contrast to settling economic, geographic and political disputes.

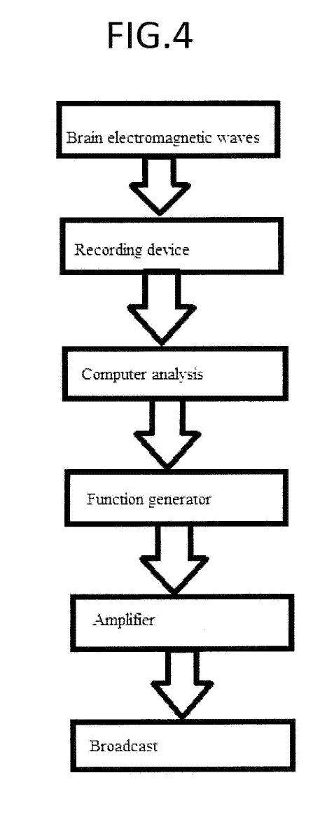

In this invention, electrical activity from the brain is recorded in an individual or a group. A computer analyzes the electrical activity with a Fourier transformation, reproducing the electrical activity with a time lag or phase shift. The reproduced electrical activity is transferred to an arbitrary function generator to generate and amplify the signal. The coherent waves are broadcast to an individual or group in the process of reconciliation. (FIG. 4)

### Benefits to Society

Negotiations are the primary method to resolve conflicts. Conflict resolution does not guarantee peace. Without reconciliation, which is a slow process that occurs in the mind, long lasting peace may not be achieved. Coherent external electromagnetic waves broadcast into a conflict may aid reconciliation by producing EEG coherence associated with a more peaceful state of mind. Peace may be more easily obtainable with this invention, but this invention will not in itself solve the reconciliation problem.

### REFERENCES

Colemen, P. T., Deutsch, M. (2015). Morton Deutsch: A Pioneer in Developing Peace Psychology: Springer International Publishing

Ferguson, P. C. (1975). Psychobiology of Transcendental Meditation—Review. Journal of Altered States of Consciousness, 2(1), 15-36.

Goldberg, J. S. (2008). Global Tramatic Stress: Hypothetical Events and Possible Solutions. Traumatology, 20(10), 1-3.

Goldberg, J. S. (2015). THERMODYNAMIC MODEL OF A NERVOUS SYSTEM. 2015/0379898 A1.

Goldberg, J. S. (2016a). METHOD TO MAINTAIN PEACE THROUGH ELECTROMAGNETIC ENERGY TARGETED TO THE BRAIN. Application Ser. No. 15/057,146.

Goldberg, J. S. (2016b). METHODE TO OPTIMIZE ELECTRODE PLACEMENT FOR CRANIAL ELECTRICAL STIMULATION. 2016/0129238 A1.

Having described my invention, I claim:

1. A method to facilitate reconciliation of past events during conflict negotiations comprising,
a. recording electromagnetic waves emitted from the brain
b. analyzing the electromagnetic waves
c. broadcasting the analyzed electromagnetic waves.

2. The method of claim 1 where the analyzed electromagnetic waves are broadcasted via air or conductive wire at a frequency between 5 and 50 hertz with a unipolar wave conformation.
